(19) United States
(12) Patent Application Publication
(10) Pub. No.: US 2014/0134672 A1
(43) Pub. Date: May 15, 2014

(54) BIOREACTORS CIRCULATION APPARATUS, SYSTEM AND METHOD

(71) Applicants: Max B. Tuttman, Cambridge, MA (US); David A. St. Angelo, Austin, TX (US)

(72) Inventors: Max B. Tuttman, Cambridge, MA (US); David A. St. Angelo, Austin, TX (US)

(21) Appl. No.: 14/130,781

(22) PCT Filed: Jul. 5, 2012
(86) PCT No.: PCT/US12/45518

§ 371 Date: Jan. 3, 2014

Related U.S. Application Data
(60) Provisional application No. 61/504,979, filed on Jul. 6, 2011.

(51) Int. Cl. C12M 1/00; C12P 7/64; C12P 7/06

(52) U.S. Cl. CPC C12M 21/02; C12P 7/065; C12P 7/649
USPC 435/47; 435/292.1; 435/161; 435/134; 435/160; 435/166; 435/158; 435/135; 435/124; 435/121; 435/139; 435/140; 435/146; 435/67; 435/144; 435/110; 435/145; 435/109; 435/116; 435/167; 435/52; 435/76; 435/69.1; 435/115; 435/123

(57) ABSTRACT

Bioreactors, and particularly, photobioreactors having a reactor chamber and surge driver, and methods for using these devices, for example, for the production of carbon-based products are provided. The reactor chamber provides a housing for microorganisms and culture medium. The surge driver produces a surge of the microorganisms and/or culture medium in the reactor chamber.

### RELATED APPLICATION(S)

This application claims the benefit of U.S. Provisional Application No. 61/504,979, filed on Jul. 6, 2011. The entire teachings of the above application(s) are incorporated herein by reference.

### BACKGROUND OF THE INVENTION

As the world's energy demands increase and energy production from non-renewable sources becomes more expensive, difficult, and harmful to the environment, the desire to capture energy from the sun has increased correspondingly.

Photobioreactors employing sunlight have been described for the production of biofuels, and other products of interest, from microorganisms. Suitable microorganisms, typically phototrophic microorganisms, are grown or propagated in photobioreactors using carbon dioxide and solar energy for the production of biomass or the production of specific compounds such as ethanol.

Previous bioreactor designs have employed process drive units such as pumps, paddlewheels, or airlift columns, in conjunction with a large surface area reactor to operate the bioreactor at substantially steady bulk flow velocity of culture medium within the reactor chamber. Difficulties of these systems can include less than desirable light-dark cycling, build up of concentration gradients, and more generally less than desirable mixing causing low system productivity. Further, use of flow rates sufficient to ameliorate one or more of these difficulties typically hinders the use of thin-film materials for containing the culture medium due to associated high hydrostatic pressures, and thereby increases costs associated with manufacturing and maintenance of the reactor chamber.

Therefore, new bioreactors and methods of operating same are needed that allow, for example, reduction of concentration gradients within the culture medium in the reactor chamber, improved light-dark cycling, and overall greater mixing thereby increasing system productivity, as well as allowing the use of thin-film materials as part of the reactor chamber to reduce reactor chamber associated costs.

### SUMMARY OF THE INVENTION

A first embodiment of the present invention is a bioreactor. The bioreactor includes a reactor chamber for housing microorganisms and culture medium, and a surge driver for producing a surge of the microorganisms and/or culture medium within the reactor chamber.

A second embodiment of the present invention is a method of producing microorganism in a bioreactor. The method includes providing microorganisms and/or culture medium in a reactor chamber, and inducing a surge of the microorganisms and/or culture medium within the reactor chamber.

A third embodiment of the present invention is a photobioreactor. The photobioreactor comprises a thin-film photobioreactor chamber for housing photosynthetic microorganisms and culture medium that is at least partially transparent to light of a wavelength that is photosynthetically active in the phototrophic microorganisms, and a surge driver for producing a continuous flow with surges of the microorganisms and/or culture medium in the photobioreactor chamber wherein the surge driver comprises a pump, a sparge device, and a reservoir, the pump draws the microorganisms and/or culture medium from a first end of the photobioreactor chamber and fills the sparge device, the sparge device sparges gas through the microorganisms and/or culture medium, and an automatic valve in the reservoir intermittently release the microorganisms and/or culture medium into a second end of the photobioreactor chamber.

A fourth embodiment of the present invention is a photobioreactor. The photobioreactor includes a reactor chamber for containing phototrophic microorganisms and culture medium e. The reactor chamber is at least partially transparent to light of a wavelength that is photosynthetically active in the phototrophic microorganisms. The photobioreactor further comprises a surge driver having a pump and a reservoir. The surge driver is connected to the reactor chamber to allow flow of culture medium from the surge driver to the reactor chamber and flow from the reactor chamber to the surge driver. The reactor chamber, reservoir and flow of culture medium are adapted to produce a surge of phototrophic microorganisms and/or culture medium within the reactor chamber as a result of releasing culture medium from the reservoir into the reactor chamber or culture medium from the reactor chamber into the reservoir.

A fifth embodiment of the present invention is a method for producing a carbon based product in a photobioreactor. The method includes repeatedly releasing culture medium from a reservoir or into a reservoir to repeatedly cause a surge of a phototrophic microorganism and/or culture medium e within a reactor chamber. The reactor chamber is at least partially transparent to light of a wavelength that is photosynthetically active in the phototrophic microorganism, and the phototrophic microorganism is adapted for producing the carbon based product in the presence of i) the culture medium and ii) the light of a wavelength that is photosynthetically active in the phototrophic microorganism.

A further embodiment of the present invention is a bioreactor. The bioreactor includes a reactor chamber and a surge driver. The reactor chamber provides a housing for microorganisms and culture medium. The surge driver produces a surge of the microorganisms and/or culture medium in the reactor chamber.

Other embodiments can include one or more of the following variations. The surge can be periodic and release a controlled quantity of the microorganisms and/or culture medium. The surge driver can include a reservoir of microorganisms and culture medium that is intermittently released into the reactor chamber to produce the surge. The intermittent release can be controlled by an automatic valve. The reactor chamber can be limited to a static pressure below about 3 PSI. The reactor chamber can be a thin-film reactor photobioreactor vessel. The surge device can produce a surge of the microorganisms and/or culture medium in multiple reactor chambers of the bioreactor. The surge driver can draw the microorganisms and/or culture medium from a first end of the reactor chamber and produces the surge by release of the microorganisms and/or culture medium in a second end of the reactor chamber located upstream of a flow of the microorganisms and/or culture medium in the reactor chamber.

In another embodiment, the surge driver can include a sparge device and a reservoir. The sparge device sparges gas through the microorganisms and/or culture medium removing excess oxygen from the culture. A valve in the reservoir intermittently releases the microorganisms and/or culture medium into the reactor chamber. In yet another embodiment, the surge driver can include a pump, a sparge device, and a reservoir. The pump can draw microorganisms and/or culture medium from a first end of the reactor chamber and fills the sparge device. The sparge device can sparge gas through the microorganisms and/or culture medium. The pump can cause the microorganisms and/or culture medium to flow into the reservoir. A valve in the reservoir intermittently releases the microorganisms and/or culture medium into a second end of the reactor chamber.

The present invention is not intended to be limited to a system or method that must satisfy one or more of any stated objects or features of the invention. It is also important to note that the present invention is not limited to the exemplary or primary embodiments described herein. Modifications and substitutions by one of ordinary skill in the art are considered to be within the scope of the present invention.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a profile diagram of a surge circulation bioreactor constructed in accordance with an exemplary embodiment of the invention.

FIG. 2 is a profile diagram of an automatic float valve of a surge driver constructed in accordance with an exemplary embodiment of the invention.

FIG. 3 shows profile diagrams of an automatic flush valve of a surge driver in closed state (A) and open flow state (B) according to an exemplary embodiment of the invention.

FIG. 4 shows profile diagrams of a surge circulation bioreactor constructed in accordance with an exemplary embodiment of the invention during three different operating stages: reservoir refilling state (A), sparging state (B), and reservoir release/surge state (C).

FIG. 5 shows profile diagrams of a surge driver having a reservoir including a sparge device and a distribution header, allowing flow of culture medium to a plurality of reactor chambers (e.g., channels, or capsules) constructed in accordance with an exemplary embodiment of the invention during four different operating stages: reservoir refilling and sparge state (A), reservoir release and sparge state (B), and completion of reservoir release (C), and distribution header release (D).

FIG. 6 is a profile diagram of surge circulation bioreactor constructed in accordance with another exemplary embodiment of the invention.

FIG. 7 provides results of a computational fluid dynamic simulation of a surge circulation bioreactor in accordance with an exemplary embodiment of the invention.

FIG. 8 provides a schematic, cross-sectional side view of an exemplary bioreactor of the present invention, in which a controlled volume of culture medium and/or microorganisms is released by volume displacement with a gas, leading to a surge of culture medium and/or microorganisms through the reactor chamber.

The drawings are not necessarily to scale, emphasis instead being placed upon illustrating embodiments of the present invention.

### DETAILED DESCRIPTION OF THE INVENTION

A description of preferred embodiments of the invention follows. The relevant teachings of all patents, published applications and references cited herein are incorporated by reference in their entirety.

The following explanations of terms and methods are provided to better describe the present invention and to guide those of ordinary skill in the art in the practice of the present invention. As used herein, “comprising” means “including” and the singular forms “a” or “an” or “the” include plural references unless the context clearly dictates otherwise. For example, reference to “comprising a phototrophic microorganism” includes one or a plurality of such phototrophic microorganisms. The term “or” refers to a single element of stated alternative elements or a combination of two or more elements, unless the context clearly indicates otherwise.

Unless explained otherwise, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. The materials, methods, and examples are illustrative only and not intended to be limiting. Other features of the invention are apparent from the following detailed description and the claims.

The surge device and reactor chamber according to embodiments of the invention can release controlled volumes of fluid into a reactor chamber in a controlled volume and/or low pressure manner. The controlled volume can be provided by part or all of the volume of a reservoir that can be refilled by a pump pulling culture medium from the outlet of the reactor chamber, or from a spillover collection that collects culture medium as it overflows through the outlet of the reactor chamber. The controlled volume can be released by, for example, an electromechanical device, such a solenoid valve, by a fluid mechanically actuated device such as a buoyancy actuated valve, or by volume displacement by a fluid (typically, gas such as air or carbon dioxide) of different (typically, lower) density.

Previous reactor designs have employed a process drive unit, such as a pump, paddlewheel, or airlift column, in conjunction with a large surface area reactor; however these systems run at a steady bulk flow velocity. In embodiments of the invention, a liquid of microorganisms and/or culture medium in the reactor chamber can cycle between periods of high flow and relative stagnation. These devices and methods of operation can provide several benefits. By having periods of high flow, greater mixing in the system can be achieved, thereby increasing system productivity by breaking up concentration gradients as well as increasing light-dark cycling of the microorganisms. Embodiments of the invention can provide mixing equal to mixing of higher continuous flow rates without the significantly larger pumping loads required for the higher continuous flow rates. Embodiments of the invention can also facilitate the use of thin film reactors because the system is not subjected to elevated pressures, as can be the case in a pumped system. In addition, a single control volume can be utilized to distribute flow to a number of reactor chambers, as it can be filled and then released into each discrete reactor chamber simultaneously. Thus, embodiments can also be used to overcome the challenge of maldistribution faced when attempting to run a pumped system through parallel reactor chambers.

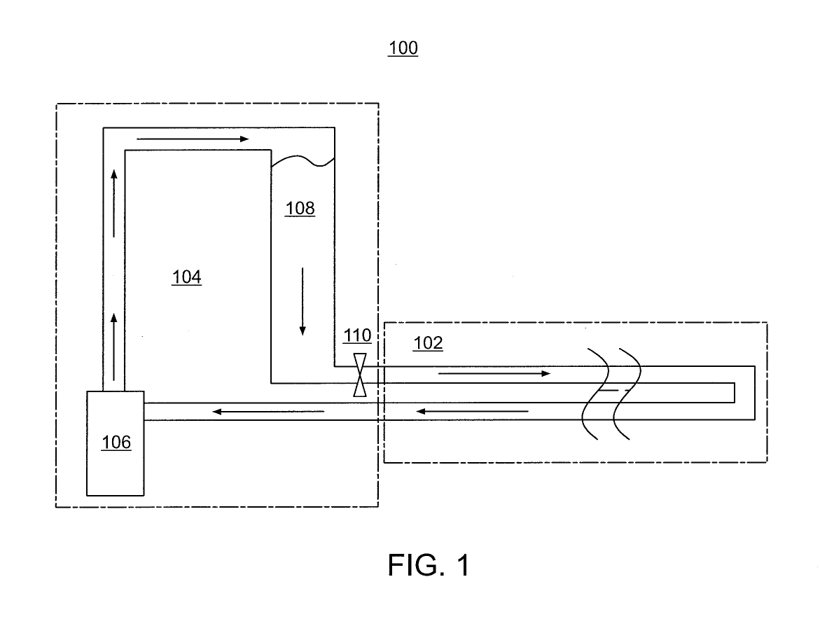

A bioreactor 100, as shown in FIG. 1, includes a substantially horizontally oriented elongated reactor chamber 102 for circulation of a culture medium and/or microorganisms driven by a surge driver 104. The surge driver 104 can include a pump 106 for filling a reservoir 108. The pump 106 draws microorganisms and/or culture medium from an exit port of the reactor chamber 102 and fills the reservoir 108 partly or completely. A valves 110 couples the reservoir 108 to the reactor chamber 102 intermittently allowing the flow of the microorganisms and/or culture medium into an entrance port of the reactor chamber 102. The intermittent releases result in surges of flow of microorganisms and/or culture medium in the bioreactor from the entrance port to the exit port. The surges of flow also can provide improved and/or more efficient mixing of the culture medium and/or microorganism. The valve 110 can be controlled by an electromechanical device, such as a solenoid valve. The solenoid can actuate the valve at predetermined periods of time to allow for a controlled volume of culture medium and microorganisms to pass through the valve 110 and enter the reactor chamber 102. Vents (not shown) can also be incorporated in the reactor chamber 102 and/or reservoir 108 to prevent a negative pressure buildup and thus reduce the flow of surges. Other valve assemblies can use mechanically actuated valves, for example, fluid mechanically actuated devices such as a buoyancy actuated valve.

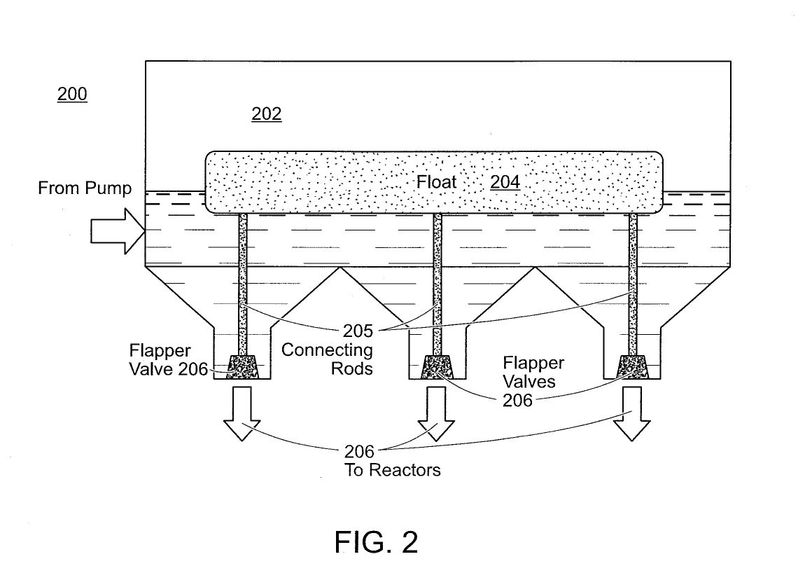

Referring to FIG. 2, an exemplary automatic float valve of as part of a surge driver 200 is provided in accordance with an exemplary embodiment of the invention. Culture medium and microorganisms from a pump fill the reservoir 202 (e.g., a reservoir 108 as shown in FIG. 1). Once the level of culture medium 203 fills the reservoir to a predetermined level, a float 204 in the reservoir 202 raises and actuates valves 206 (here three valves are shown, generally one or more can be used) through connecting rods 205, connecting the reservoir 202 to one or more reactor chambers (e.g., a reactor chamber 102 as shown in FIG. 1). In the embodiment shown in FIG. 2, the reservoir 202 includes outlets for three reactor chambers 110. The culture medium and microorganisms flow out of the reservoir 202 and into each of the three reactor chambers (not shown in FIG. 2). The valves 206 can be of a flush type that opens when activated by the float and are maintained in an open position by the rapid flow of fluid through the valve. As the fill level in the reservoir 202 decreases to a controlled or predetermined level, the flow through the valve reduces causing the valve to close and allow the pump to fill the reservoir 108 for one or more subsequent surge cycles.

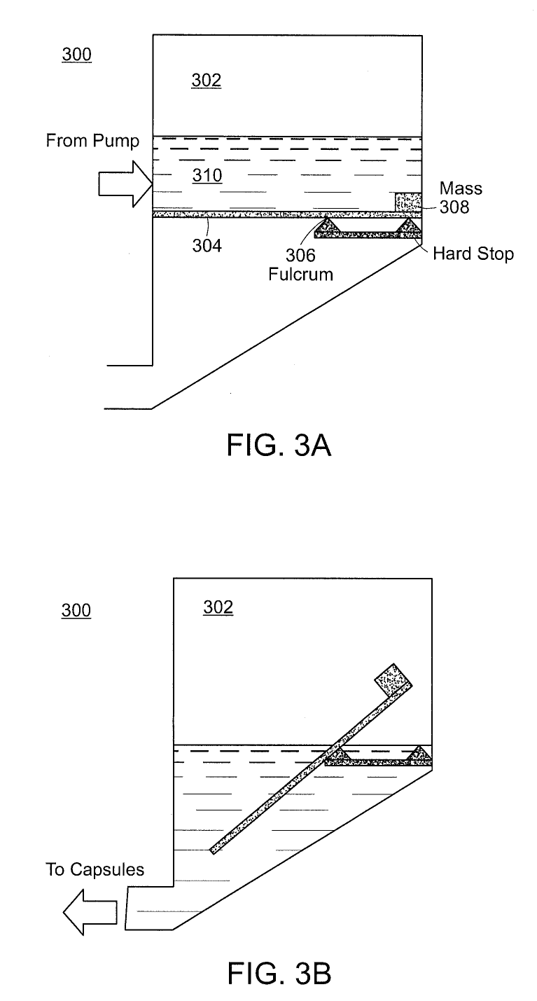

Referring to FIG. 3, an exemplary automatic flush valve 300 of a surge driver (e.g. 104) is provided in closed state (A) and open flow state (B). When the valve is in the closed state (A), Culture medium and microorganisms from a pump (e.g. 106) fill the reservoir 302 (which can be reservoir 108 in FIG. 1) (as shown in FIG. 3A). Once the level of fluid fills the reservoir to a controlled or predetermined volume, the weight of the culture medium and microorganisms causes the lever 304 to rotate about the fulcrum 306 and lift a counter mass 308 leading to the open flow state B. Rotation of the lever 304 in this manner opens the valve and allows culture medium 310 to flow out of the reservoir, for example, into one or more reactor chambers or into a distribution header (not shown). As the reservoir 302 empties and the flow through the valve is sufficiently reduced, the force of the fluid in the reservoir 302 on the lever is reduced causing the lever to rotate around the fulcrum 306 leading to the closed valve state as shown in A. The closed valve allows the pump to fill the reservoir 302 for one or more subsequent surge cycles. Exemplary embodiments are not limited to the above-described valve configurations and can use other valve configuration that allow for a surge of flow into or out of the reactor chamber.

The one or more reactor chambers of the bioreactor can enclose a phototrophic microorganism, such as algae or cyanobacteria. Phototrophic organisms growing in photobioreactors can be suspended or immobilized. Prototrophic microorganisms contained in photobioreactors require light to grow and/or produce carbon-based products of interest. Therefore, photobioreactors, and in particular reactor chambers are adapted to provide light of a wavelength that is photosynthetically active in the phototrophic microorganism to reach the culture medium. Typically, at least part of the reactor chamber can be transparent for light of a wavelength that is photosynthetically active in the phototrophic microorganism. This can be achieved by proper choice of the material for the reactor chamber, for example thin-film material, to allow light to enter the interior reactor chamber 102.

Reactor chambers can include or be provided by a thin-film material enclosure, typically made from a polymeric material. The reactor chamber can include a headspace which allows, for example, flowing of carbon dioxide for the phototrophic microorganism and, generally, gas flow (e.g., for providing the gas to the microorganism, for cooling or for other purposes). The bioreactors can include one or more reactor chambers. The bioreactor chambers can be of different shapes (e.g., elongated semi-circle shaped, flat, etc.) and sizes. The reactor chambers can be, for example, relatively shallow and/or flat. Shallow elongated reactor chambers can be advantageous in increasing the mixing of the microorganisms and culture medium effected by the surges. This can also be advantageous, for example, for positioning of the photobioreactors on flat surfaces such as flat ground or a body of water, for example, a lake. In other embodiments, the reactor chamber can be at a slight slope to assist the surges in producing an overall flow from the entrance port to the exit port of the reactor chamber.

The reactor chamber(s) of the photobioreactor can be adapted to allow cultivation of the phototrophic microorganisms in a thin layer. Typically, the layer in the absence of a surge is between about 5 mm and about 30 mm thick, or, more typically, between about 10 mm and about 15 mm.

The reactor chamber(s) can be thin-film, shallow elongated reactor chambers. Typically, the reactor chambers are characterized in operation by having an average (taken along the width and length of the reactor chamber) height in cross-section of at least about 10 mm, more typically, of at least 15 mm, even more typically, between about 10 mm and about 100 mm, and yet even more typically, between about 15 mm and about 50 mm.

The reactor chambers can be made of a polymeric material that is limited to low hydrostatic pressures. Typically, this refers to a hydrostatic pressure of less than about 5 PSI, and more typically, less than about 3 PSI.

The bioreactor 100 can include further elements (not shown) such as inlets and outlets, for example, for growth media, carbon sources (e.g., CO2), and probe devices such as optical density measurement devices and thermometers. Heat exchange chambers and other chambers can be incorporated into embodiments of the invention. Alternatively, the bioreactor(s) can also be placed above the ground and can utilize solid support structures, for example, made of metal, mesh or fabric. The bioreactors can be operated in batch or continuous mode.

Bioreactors of various sizes can be suitable for exemplary embodiments of the present invention. Bioreactor size can be influenced by the material and manufacturing choices. For example, in some embodiments of the present invention, the reactor chambers are made of a thin film polymeric material which can be, for example, between 1 and 100 meters long. In some embodiments, the reactor chamber is 40 meters long. A further consideration is transportability of a manufactured bioreactor, which can be greatly enhanced by using flexible thin-film. Bioreactor chambers can be designed to be folded at least to some extent and/or rolled for more compact storage. For bioreactors with very large bioreactor chambers this is a significant advantage, because it can prevent costly transportation permits and oversized transport vehicles, or alternatively, significant installation costs at the installation site.

Each reactor chamber of the bioreactor can be of a different shape and dimension. The flow through the individual valves can be designed for various reactor chamber designs or onsite conditions, for example, the amount of culture medium used to produce the surge can be increased or decreased by on the onsite slope of the reactor chambers, however, typically, the reactor chamber(s) are substantially horizontally (i.e., including some tilt or unevenness which can be due to the unevenness of the ground, or otherwise provided) positioned. In another example, multiple reactor chambers can be of different sizes with individual valves designed for different surge flows. Typically, however, in bioreactors with a plurality of reactor chambers, the reactor chambers are of similar or identical shape and dimensions, for example, channels positioned in parallel with substantially longer channel length than width with identical valves controlling the flow into each identical reactor chamber. Various reactor chamber cross-sections are suitable, for example, rectangular, cylindrical, or half-elliptical. Preferably, the reactor chamber is half-elliptical or rectangular. Further, reactor chamber(s) can be enclosures (e.g., bags) welded from thin polymeric films. Such reactor chambers can allow for advantageous compact transport, facilitate sterilization (e.g., with radiation such as gamma radiation) prior to deployment, and allow use as disposable reactor chamber(s) because of the cost-efficiency and/or energy efficiency of their production. They can also be reused.

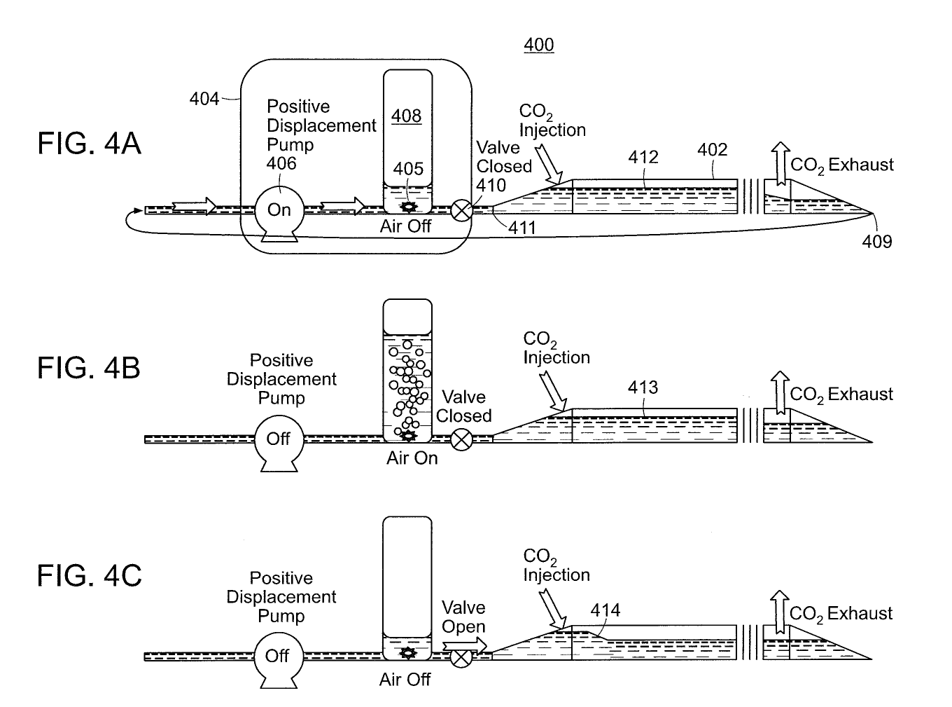

Referring to FIG. 4, an exemplary bioreactor 400 is shown including a substantially horizontally oriented elongated reactor chamber(s) 402 adapted for circulation of culture medium and/or microorganisms 403 driven by a surge driver 404 having a sparge device 405. The surge driver 404 can include a pump 406 for pumping culture medium and/or microorganisms 403 into a reservoir 408. The pump 406 draws microorganisms and/or culture medium 403 from an exit port 409 of the reactor chamber(s) 402 and fills the reservoir 408. The sparge device 405 allows sparging of a strip gas such as carbon dioxide or air into the reservoir 408. This can be used to reduce the levels of oxygen and increase the levels of carbon dioxide in the microorganisms and/or culture medium 403. One or more valve 410 that couple the reservoir 408 to the reactor chamber(s) 402 intermittently allow the flow of the microorganisms and/or culture medium 403 into an entrance port 411 of the reactor chamber 402. During stage (A), the pump 406 is on, that is, pumping microorganisms and/or culture medium 403 into the reservoir 408, which fills the reservoir because valve(s) 410 are closed. During this stage the reactor chamber(s) 402 slowly release (typically, without a surge) microorganisms and/or culture medium 403 such that the fluid fill level within the reactor chamber(s) decreases from a first level 412 to a lower second level 413. While the reservoir fills with microorganisms and/or culture medium 403, gas 414 can be sparged through the microorganisms and/or culture medium 403 (not shown as separate diagram). Further, once the reservoir is filled to a controlled or predetermined level as shown in B, the pump 406 can be turned off and the sparging of gas started or continued to strip oxygen and/or increase the concentration of carbon dioxide in the culture medium contained within the reservoir. When the valve(s) 410 are opened, microorganisms and/or culture medium 403 is released into the reactor chamber(s) 402 leading to a surge 414 of microorganisms and/or culture medium within the reactor chamber and a subsequent increase of the fill level back to a level similar or substantially identical to the prior fill level 412 shown in A. The intermittent releases result in surges of flow of microorganisms and/or culture medium in the bioreactor from the entrance port to the exit port. The surges of flow also can provide improved and/or more efficient mixing of the culture medium and/or microorganisms. The components of the bioreactor 400 can incorporate structures and various features as described in prior embodiments.

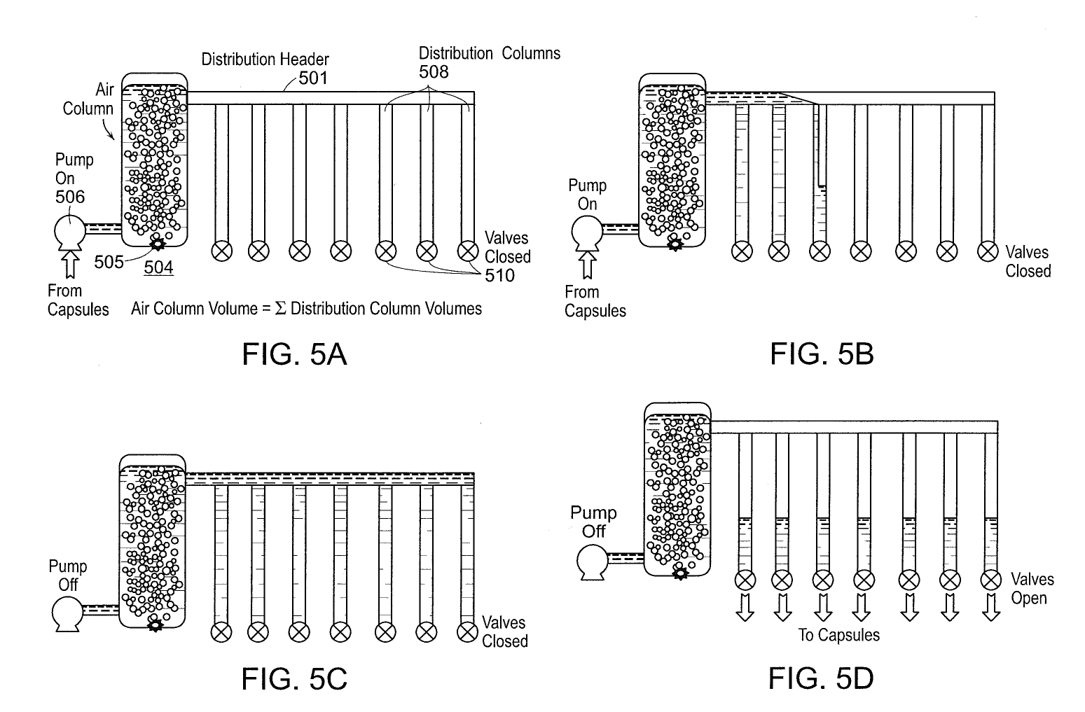

FIG. 5 provides an exemplary surge driver 500 for use in a bioreactor. The surge driver 500 includes a distribution header 501 connected to a reservoir 505 having a sparge device 505. The surge driver 500 can include a pump 506 for filling the reservoir 504. The pump 506 draws microorganisms and/or culture medium from an exit port of the reactor chamber(s) (not shown) and fills the reservoir 504 as shown in A. The gas sparge device 505 can release gas into the riser section through a gas compressor or other gas input device. The sparge device 505 can be used to reduce the levels of oxygen and increase the levels of carbon dioxide in the microorganisms and/or culture medium by, for example, using carbon dioxide as a sparge gas. The pump 506 can further pump microorganisms and/or culture medium from the reservoir 504 into the distribution header 501 and its individual reservoirs 508 as shown in stage B and C. Valves 510 that couple the individual reservoirs 508 to the reactor chamber(s) allow control of the flow of the microorganisms and/or culture medium into an entrance port of the reactor chamber(s). For example, in stage D, all of the valves 510 are open and all of the reservoirs 508 are being released into the reactor chamber(s) to produce one or more surges within the reactor chambers. The intermittent releases result in surges of flow of microorganisms and/or culture medium in the bioreactor from the entrance ports to the exit ports. The surges of flow also can provide improved and/or more efficient mixing of the culture medium and/or microorganisms. The components of the bioreactor 500 can incorporate structures and various features as described in prior embodiments.

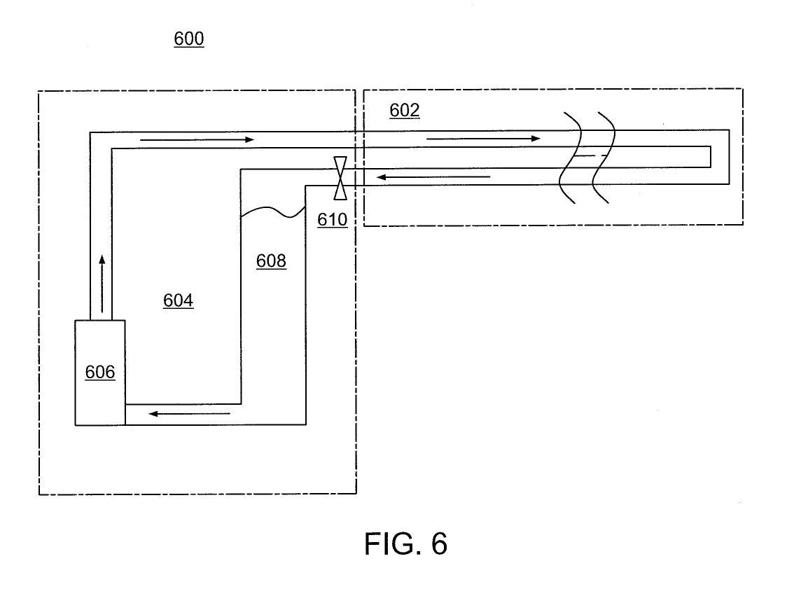

Referring to FIG. 6, a bioreactor 600 includes a reactor chamber 602 with a circulation of culture medium and/or microorganisms driven by a surge driver 604 by drawing surges of fluid from the reactor chamber 602. The surge driver 604 can include a pump 606 that fills the reactor chamber 602 from a reservoir 608 in a continuous manner. A valve 610 couples an exit port of the reactor chamber 602 to the reservoir 608. The valve 610 intermittently allows the flow of the microorganisms and/or culture medium into the reservoir 608 from the exit port of the reactor chamber 602. The intermittent releases result in surges of flow of microorganisms and/or culture medium in the bioreactor from the exit port to an entrance port. The surge in flow can be provided by the valve 610 having a large flow surface area being actuated for short intervals of time. In another example, the reactor chamber 602 can be pressurized resulting in a surge when the substantial flow when the valve 610 is opened. The surges of flow also can provide improved and/or more efficient mixing of the culture medium and/or microorganisms. The components of the bioreactor 600 can incorporate structures and various features as described in prior embodiments.

Typical embodiments utilize an inflow surge into the reactor chamber over a draw surge of the reactor chamber, described in the prior embodiment. In, another embodiment of the invention, the surge device can include a device that compresses or elevates a portion of the reactor chamber to result in a surge or wave of the microorganisms and/or culture medium within the reactor chamber. In addition, a continuous flow can also be provided in conjunction with the intermittent surges.

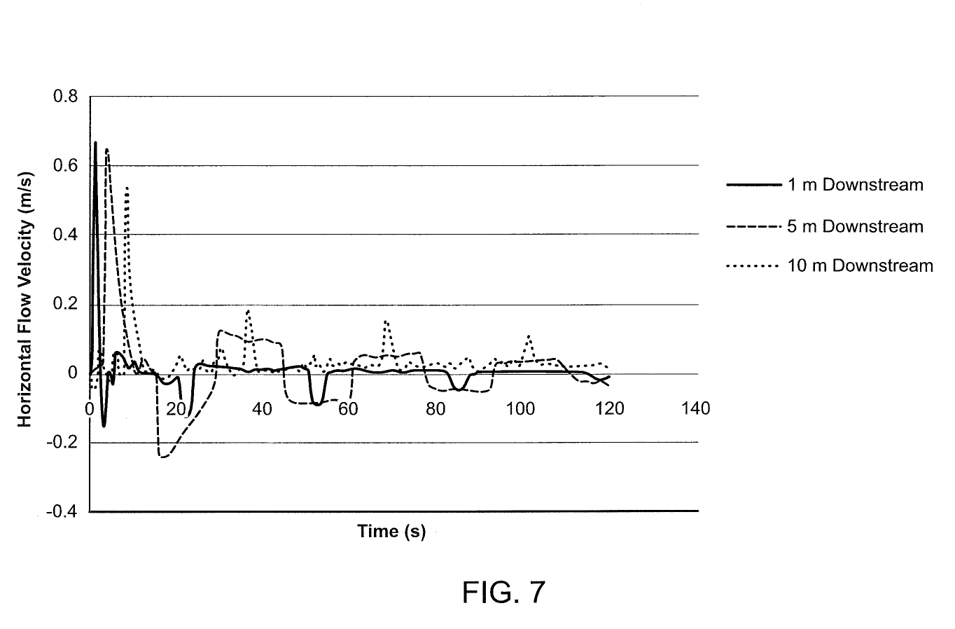

Referring to FIG. 7, a computational fluid dynamic simulation of a surge flow velocity is provided for a simulated bioreactor in accordance with an exemplary embodiment of the invention. The simulation is a two dimensional Volume of Fluid simulation with a fluid of 1060 kg/m³ (density) and 0.002 N*s/m² (viscosity). The simulated volume of fluid released was 1.5 m high and 0.1 m wide into a simulated reactor vessel of 10 m long and outlet weir height of 30 mm. Horizontal flow velocities were provided at 1 m, 5 m, and 10 m downstream.

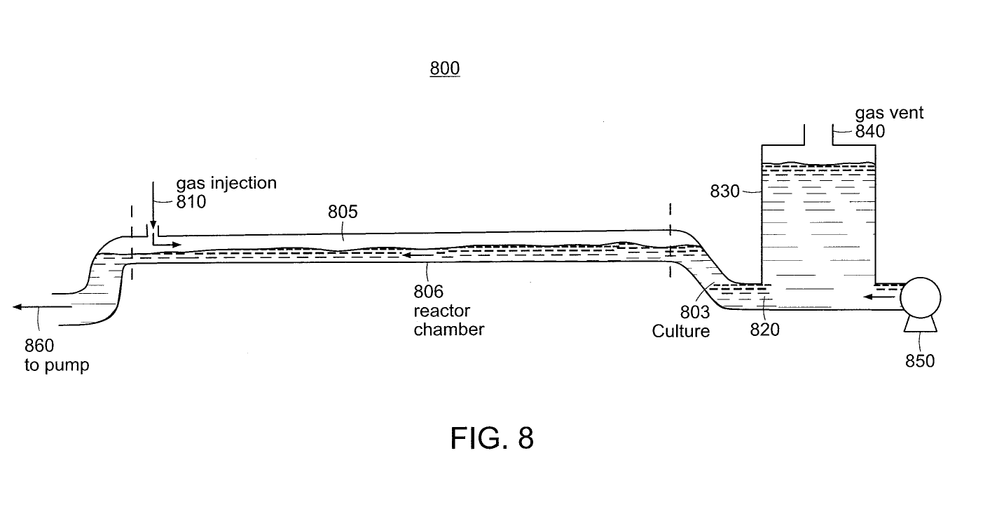

FIG. 8 provides a schematic, cross-sectional side view of an exemplary bioreactor 800 of the present invention. In this embodiment, volume displacement of the culture medium and/or microorganisms 803 (referred to as culture) by a second fluid of different density, typically a gas, such as air or CO₂, leads to a surge of the culture fluid as described in the following. As gas 805 is (typically, continuously) injected into the reactor chamber 806 (typically, through an gas injection inlet 810 of the reactor chamber 806), the gas 805 begins to displace the culture 803 (by first forming a gas space above the culture 803), and continues to displace the culture 803 until the gas 805 has driven the culture 803 down low enough to expose (typically, partically) the opening of the reservoir 830 (typically, a tank). At this point, gas, traveling counter flow to the culture, is able to escape from the reactor chamber 803 through the opening 820, into the tank 830 and up and through the vent 840 which is typically located in the top of the tank 820. When this occurs, a surge of culture, coming from the tank 830, will enter the reactor chamber 806 through opening 820, replacing the volume vacated by the gas. Culture 303 is pumped into the reservoir 830 with a pump 850 in fluid communication with the reservoir 830. After occurrence of the surge of culture through the reactor chamber 806, culture can flow through reactor chamber outlet 860. From there it can be circulated and pumped back into reservoir 830, for example, with pump 850.

Microorganism Production

Typically, the bioreactors referred to herein are photobioreactors. Suitable photobioreactors and culture media for cultivating phototrophic microorganisms are known in the art. Particularly suitable culture media are described in U.S. application Ser. No. 13/061,116, filed Dec. 11, 2009.

Typically, the culture medium is liquid. More typically, it is an aqueous liquid including nutrients suitable for the phototrophic microorganism(s) that are being cultivated in the photobioreactor(s).

The bioreactors described herein also provide the basis for methods to achieve organism productivity as measured by production of desired products, which includes cells themselves.

The desired level of products produced from the engineered light-capturing organisms in the photobioreactor can be of commercially utility. For example, the engineered light-capturing organisms in the photobioreactor convert light, water and carbon dioxide to produce fuels, biofuels, biomass or chemicals at about 5 to about 10 g/m²/day, in certain embodiments about 10 to about 30 g/m²/day and in more preferred embodiments, about 30 to 45 g/m²/day or greater.

The photobioreactor system affords high areal productivities that offset associated capital costs. Superior areal productivities are achieved by: optimizing cell culture density through control of growth environment, optimizing CO₂ infusion rate and mass transfer, optimizing mixing to achieve highest photosynthetic efficiency/organisms, achieving maximum extinction of insulating light via organism absorption, achieving maximum extinction of CO₂.

In particular, the southwestern U.S. has sufficient solar insulation to drive maximum areal productivities to achieve about >25,000 gal/acre/year ethanol or about >15,000 gal/acre/year diesel, although the majority of the U.S. has insulation rates amenable to cost-effective production of commodity fuels or high value chemicals.

Furthermore, CO₂ is also readily available in the southwestern U.S. region, which is calculated to support large-scale commercial deployment of the invention to produce 120 Bn gal/year ethanol, or 70 Bn gal/year diesel.

Exemplary Setup

One setup of the surge reactor system includes a surge volume constructed of 1½″ PVC pipe, a hand operated PVC ball valve for actuation, a hand operated diaphragm pump, and a 4″ flat width low-density polyethylene tube with machined connectors, acting as the reactor chamber. Air is injected into the reactor chamber at its inlet, just downstream of the surge volume. A CO₂ rich gas can also be injected into the reactor volume. A vent can be located just prior to the outlet of the capsule, allowing for the air to escape. Liquid can be pumped from the outlet of the capsule into the surge volume. When the surge volume reaches a desired level, the ball valve can be turned from the closed position to the open position, allowing for the liquid to rush from the surge volume into the reactor chamber. The ball valve can then be returned to the closed position in order to allow for the filling of the surge volume.

Another setup of the surge reactor system can employ a surge volume made from a polycarbonate container, a flush valve actuator, an electric diaphragm pump, and a 0.4 m flat width low-density polyethylene tube with welded in sanitary connections, provided by ATMI. A buoy can be connected with a chain to the flush valve, such that when the liquid level in the container reached a desired height, the valve would open, releasing the liquid into the reactor. Air can be injected just prior to the inlet of the capsule, and allowed to escape through a vent just prior to the outlet. The diaphragm pump draws liquid from the outlet of the reactor, filling the container, until the flush valve opened. The pump can be run both continuously, pouring liquid into the surge volume even while the surge was occurring, and intermittently, allowing for the surge to complete before refilling the volume.

### DEFINITIONS

Suitable phototrophic microorganisms can produce a carbon-based product and/or the phototrophic microorganism itself can be processed as feed stock for the production of a carbon-based product. Particularly suitable phototrophic microorganisms can be genetically engineered to produce a desired carbon-based product. Exemplary suitable phototrophic microorganisms are described in U.S. Pat. No. 7,919,303, U.S. Pat. No. 7,794,969, U.S. patent application Ser. No. 12/833,821, U.S. patent application Ser. No. 13/054,470, U.S. patent application Ser. No. 12/867,732, WO/2009/111513, WO/2009/036095, WO/2011/005548, WO/2011/006137 and WO/2011/011464.

“Carbon-based products” include alcohols such as ethanol, propanol, isopropanol, butanol, fatty alcohols, fatty acid esters, ethyl esters, wax esters; hydrocarbons and alkanes such as pentadecane, heptadecane, propane, octane, diesel, Jet Propellant 8 (JP8); polymers such as terephthalate, 1,3-propanediol, 1,4-butanediol, polyols, Polyhydroxyalkanoates (PHA), poly-beta-hydroxybutyrate (PHB), acrylate, adipic acid, 8-caprolactone, isoprene, caprolactam, rubber; commodity chemicals such as lactate, docosahexaenoic acid (DHA), 3-hydroxypropionate, γ-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, 3-hydroxypropionic acid (HPA), lactic acid, THF, gamma butyrolactone, pyrrolidones, hydroxybutyrate, glutamic acid, levulinic acid, acrylic acid, malonic acid; specialty chemicals such as carotenoids, isoprenoids, itaconic acid; pharmaceuticals and pharmaceutical intermediates such as 7-aminodeacetoxycephalosporanic acid (7-ADCA)/cephalosporin, erythromycin, polyketides, statins, paclitaxel, docetaxel, terpenes, peptides, steroids, omega fatty acids and other such suitable products of interest. Such products are useful in the context of biofuels, industrial and specialty chemicals, and as intermediates used to make additional products, such as nutritional supplements, neutraceuticals, polymers, paraffin replacements, personal care products and pharmaceuticals. More typical carbon-based products are fuels or chemicals. Even more typically, carbon-based products are ethanol, propanol, isopropanol, butanol, terpenes, alkanes such as pentadecane, heptadecane, octane, propane, fatty acids, fatty esters, fatty alcohols, olefins or diesel.

Particularly suitable reactor chambers are described in U.S. application Ser. No. 13/061,116, filed Dec. 11, 2009. Other particularly suitable reactor chambers are described in U.S. application Ser. No. 13/128,365, filed Jul. 28, 2010.

As used herein, “light of a wavelength that is photosynthetically active in the phototrophic microorganism” refers to light that can be utilized by the microorganism to grow and/or produce carbon-based products of interest, for example, fuels including biofuels.

“Phototrophs” or “photoautotrophs” are organisms that carry out photosynthesis such as, eukaryotic plants, algae, protists and prokaryotic cyanobacteria, green sulfur bacteria, green non-sulfur bacteria, purple sulfur bacteria, and purple non-sulfur bacteria. Phototrophs include natural and engineered organisms that carry out photosynthesis and hyperlight capturing organisms.

The photobioreactors of the present invention are adapted to support a biologically active environment that allows chemical processes involving photosynthesis in organisms such as phototrophic organisms to be carried out or biochemically active substances to be derived from such organisms. The photobioreactors can support aerobic or anaerobic organisms.

As used herein, “microorganisms” encompasses autotrophs, phototrophs, heterotrophs, engineered light capturing organisms and at the cellular level, e.g., unicellular and multicellular.

As used herein, “light” generally refers to sunlight but can be solar or from artificial sources including incandescent lights, LEDs fiber optics, metal halide, neon, halogen and fluorescent lights.

“Intermittent” as used herein, refers to a regular or irregular periods of two or more occurrences, which is not limited to a continuous routine pattern and can occur with sporadic, random, or erratic occurrences.

Throughout this specification and claims, the word “comprise,” or variations such as “comprises” or “comprising,” will be understood to imply the inclusion of a stated integer or group of integers but not the exclusion of any other integer or group of integers.

The foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of this invention are presented for purposes of illustration and description. They are not intended to be exhaustive or to limit the invention to the precise forms disclosed; obviously many modifications and variations are possible in view of the above teachings. The embodiments were chosen and described in order to best explain the principles of the invention and its practical applications. These procedures will enable others, skilled in the art, to best utilize the invention and various embodiments with various modifications. It is intended that the scope of the invention be defined by the following claims and their equivalents. Modifications and substitutions by one of ordinary skill in the art are considered to be within the scope of the present invention, which is not to be limited except by the following claims.

While this invention has been particularly shown and described with references to example embodiments thereof, it will be understood by those skilled in the art that various changes in form and details can be made therein without departing from the scope of the invention encompassed by the appended claims.

1.-22. (canceled)

23. A photobioreactor comprising:
a thin-film photobioreactor chamber for housing photosynthetic microorganisms and culture medium that is at least partially transparent to light of a wavelength that is photosynthetically active in the phototrophic microorganisms; and
a surge driver for producing a continuous flow with surges of the microorganisms and/or culture medium in the photobioreactor chamber wherein the surge driver comprises a pump, a sparge device, and a reservoir, the pump draws the microorganisms and/or culture medium from a first end of the photobioreactor chamber and fills the sparge device, the sparge device sparges gas through the microorganisms and/or culture medium, and an automatic valve in the reservoir intermittently release the microorganisms and/or culture medium into a second end of the photobioreactor chamber.

24. The photobioreactor of claim 23, wherein the surge is periodic and with a controlled quantity of the microorganisms and/or culture medium.

25. The photobioreactor of claim 23, wherein the surge device produces a surge of the microorganisms and/or culture medium in multiple photobioreactor chambers of the bioreactor.

26. The photobioreactor of claim 23, wherein the surge driver draws the microorganisms and/or culture medium from a first end of the photobioreactor chamber and produces the surge by release of the microorganisms and/or culture medium in a second end of the photobioreactor chamber located upstream of the flow of the microorganisms and/or culture medium in the photobioreactor chamber.

27. The photobioreactor of claim 23, wherein the sparge gas includes carbon dioxide.

28. (canceled)

29. A photobioreactor comprising:
a reactor chamber for containing phototrophic microorganisms and culture medium, the reactor chamber being at least partially transparent to light of a wavelength that is photosynthetically active in the phototrophic microorganisms;
a surge driver including a pump and a reservoir; the surge driver being connected to the reactor chamber to allow flow of culture medium from the surge driver to the reactor chamber and flow from the reactor chamber to the surge driver; the reactor chamber, reservoir and flow of culture medium being adapted to produce a surge of phototrophic microorganisms and/or culture medium within the reactor chamber as a result of releasing culture medium from the reservoir into the reactor chamber or culture medium from the reactor chamber into the reservoir.

30. The photobioreactor of claim 29, wherein the surge driver is adapted to control flow rate and/or flow amount of culture medium into and/or out of the reactor chamber.

31. The photobioreactor of claim 29, wherein the pump is connected to the reservoir to allow pumping culture medium into the reservoir, and the reservoir is positioned relative to the reactor chamber such that the surge of microorganisms and/or culture medium within the reactor chamber is produced as a result of releasing culture medium from the reservoir into the reactor chamber.

32. The photobioreactor of claim 29, wherein the pump is connected to the reactor chamber to pump culture medium into the reactor chamber, and the reservoir is positioned relative to the reactor chamber such that the surge of microorganisms and/or culture medium within the reactor chamber is produced as a result of releasing culture medium from the reactor chamber into the reservoir.

33. The photobioreactor of claim 29, wherein the reactor chamber is an elongated reactor chamber made at least in part from a thin polymer film.

34. The photobioreactor of claim 29, wherein the surge driver comprises a sparge device which allows sparging of a gas through culture medium contained within the reservoir.

35-37. (canceled)

38. A method for producing a carbon based product in a photobioreactor comprising:
repeatedly releasing culture medium from a reservoir or into a reservoir to repeatedly cause a surge of a phototrophic microorganism and/or culture medium within a reactor chamber; wherein the reactor chamber is at least partially transparent to light of a wavelength that is photosynthetically active in the phototrophic microorganism, and the phototrophic microorganism is adapted for producing the carbon based product in the presence of i) the culture medium and ii) the light of a wavelength that is photosynthetically active in the phototrophic microorganism.

39. The method of claim 38, further comprising pumping culture medium into the reactor chamber for release of culture medium from the reactor chamber to cause the surge, or pumping culture medium into the reservoir for release of culture medium from the reservoir to cause the surge.

40. The method of claim 38, further comprising controlling rate and/or volume of culture medium release.

41. The method of claim 38, further comprising opening a valve to release the culture medium.

42. The method of claim 38, wherein the phototrophic microorganism and culture medium therefor is continuously present within the reactor chamber.

43. The method of claim 38, further comprising continuously flowing the phototrophic microorganism and culture medium therefor through the reactor chamber.

44. The method of claim 38, further comprising sparging a gas through culture medium within the reservoir.

45. The method of claim 38, further comprising stopping flow of culture medium from or into the reservoir thereby ending each release.

46. The method of claim 38, further comprising controlling flow rate and amount of culture medium within the reactor chamber to control hydrostatic pressure within the reactor chamber.

47-49. (canceled)
